(19) United States
(12) Patent Application Publication
(10) Pub. No.: US 2008/0081951 A1
(43) Pub. Date: Apr. 3, 2008

(54) Inflatable retractor

(75) Inventors: William J. Frasier, New Bedford, MA (US); Timothy Beardsley, Kingston, MA (US); Connie P. Marchek, Foxboro, MA (US); Sara Dziedzic, Dorchester, MA (US); Holly R. Brideau, West Roxbury, MA (US); Sean Selover, Westport, MA (US); Nicholas Pavento, Marlboro, MA (US); Charles M. Bartish, Providence, RI (US); James Roveda, N. Attleboro, MA (US); Michael Slivka, Taunton, MA (US)

Correspondence Address: LAHIVE & COCKFIELD, LLP, ONE POST OFFICE SQUARE, BOSTON, MA, 02109-2127, US

(73) Assignee: DePuy Spine, Inc., Raynham, MA (US)

(21) Appl. No.: 11/540,781

(22) Filed: Sep. 29, 2006

(51) Int. Cl. A61B 1/32

(52) U.S. Cl. 600/207

(57) ABSTRACT

The present invention provides an inflatable retractor for providing access to a surgical site, such as a patient's spine, during a surgical process. When used in spinal surgery, the inflatable retractor allows a surgeon to operate on one or more spinal levels. The inflatable retractor includes an inflatable body defining a central cavity, wherein the body is flaccid in a non-inflated state and increasingly rigid in an inflated state. The inflatable retractor is inserted into an incision in a deflated, or partially inflated, state and then inflated once the retractor is in position. The inflation of the retractor retracts skin and muscle from the surgical site, allowing adequate visualization of the surgical site and forms a passage providing access for implants and surgical instruments to pass through the retractor and into the surgical site.

### FIELD OF THE INVENTION

The present invention relates to a device and method for accessing a surgical site during surgery, such as spinal surgery. More particularly, the present invention relates to a device and method for retracting tissue surrounding a surgical site during a surgery.

### BACKGROUND OF THE INVENTION

Spinal retraction systems are used in spinal surgery to provide access to a patient's spine by retracting and holding back tissue from a targeted surgical site beneath the skin. Current spinal retraction systems are rigid and therefore have difficulty conforming to patient anatomy. The rigid nature of such spinal retraction systems may also lead to increased danger to skin, nerves, vessels or muscle tissue, and often an inability to get good muscle retraction at a desired location. Furthermore, the rigid retractor requires a sizeable incision for insertion of the retractor into a patient. In addition, rigid retractors require a bed-mounted arm to hold the retractor in place relative to the patient's body. Such bed-mounted arms can provide an obstruction to the operating field of the surgeon and if the arm is inadvertently bumped by the surgeon, the secured retractor may be moved or dislodged.

### SUMMARY OF THE INVENTION

The present invention provides an inflatable retractor for providing access to a surgical site, such as a patient's spine, during a surgical process. When used in spinal surgery, the inflatable retractor allows a surgeon to operate on one or more spinal levels. The inflatable retractor includes an inflatable body defining a central cavity therethrough, wherein the body is flaccid in a non-inflated state and increasingly rigid in an inflated state. The inflatable retractor is inserted into an incision in a deflated state and then inflated once the retractor is in position. The inflation of the retractor retracts skin and muscle from the surgical site, allowing adequate visualization of the surgical site and forms a passage providing access for implants and surgical instruments to pass through the retractor and into the surgical site.

In accordance with a first aspect of the present invention, an inflatable retractor for retracting tissues in an incision for a surgical site is provided. The inflatable retractor comprises an inflatable body defining a central cavity therethrough, wherein the body is flaccid in a non-inflated state and increasingly rigid in an inflated state.

In one embodiment, the inflatable retractor further comprises a valve functional to maintain an inflating pressure in the inflatable body. The valve may also be used to release inflating pressure in the inflatable body.

In one embodiment, the inflatable body is generally cylindrical in shape. Thus when inflated, the retractor forms a tube having a central bore proving access to a surgical site.

In one embodiment, the inflatable body is generally conical in shape. Thus when inflated, the retractor forms a funnel having a central bore that is wider at one end then the other.

In one embodiment, the inflatable body is helical in shape and configured to be coiled upon itself to define a passage for accessing a patient. Thus when inflated, the retractor can be shaped to conform as needed to provide access to the surgical site.

In embodiments wherein the inflatable body has a general shape, support structures, such as shape memory material support structures, may be used to maintain the shape of the inflatable retractor. The retractor may also conform to the surrounding anatomy while still retracting tissue. The retractor may also have distal protrusions for retracting tissue and minimizing tissue creep.

In one embodiment, the inflatable retractor further comprises an anchor attached to the inflatable body for anchoring the inflatable body to a body part within the incision. The anchor may comprise, for example, a re-absorbable anchor, a removable biocompatible anchor, or an intermediate anchor, such as a pedicle screw or bolt.

In accordance with another embodiment, a method is provided for accessing a surgical site in a patient. The method comprises the steps of creating an incision in the patient; inserting a deflated or partially inflated inflatable retractor into the incision in the patient, the inflatable retractor comprising an inflatable body defining a central cavity therethrough, wherein the body is flaccid in a non-inflated state and increasingly rigid in an inflated state; and inflating the inflatable retractor to retract tissue surrounding the incision to create a surgical site.

In one embodiment, the method further comprises the step of preparing the incision prior to inserting the inflatable retractor. Preparing the incision may include: inserting a guide into the incision, sweeping back muscle and tissue from the surgical site, and finding a target for attaching an inflatable retractor.

In one embodiment, the step of inserting the inflatable retractor comprises using an instrument to insert the inflatable retractor into the incision. Insertion may also include attaching the inflatable retractor to a target. The retractor may contain radiopaque markers to aid in percutaneous insertion under fluoroscopy.

In some embodiments, the inflatable retractor is inflated with a gas, liquid, or combination of both. In other embodiments a hardening agent may be used to inflate the retractor that increases the rigidity of the inflated retractor.

In one embodiment, the method further comprises deflating the retractor after a surgical procedure has been performed and removing the deflated retractor from the incision.

In accordance with another aspect, a system is provided for retracting tissues in an incision for a surgical site. The system comprises an insertion tool for insertion into the incision at a surgical site; and inflatable retractor configured to be guided to a surgical site by the insertion tool, the inflatable retractor comprising an inflatable body defining a central cavity, wherein the body is flaccid in a non-inflated state and increasingly rigid in an inflated state.

In one embodiment, the insertion tool comprises a dilator and the inflatable retractor is guided to the surgical site over the dilator. In yet another embodiment, the insertion tool is a guide-wire and the inflatable retractor is guided to the surgical site over the guide-wire. In yet another embodiment the insertion tool comprises a cannula and the inflatable retractor is guided to the surgical site through the cannula

### BRIEF DESCRIPTION OF THE FIGURES

The foregoing and other objects, features and advantages of the invention will be apparent from the following description and apparent from the accompanying drawings, in which like reference characters refer to the same parts throughout the different views. The drawings illustrate principles of the invention and, although not to scale, show relative dimensions

FIG. 1A illustrates an embodiment of an inflatable surgical retractor of the invention.

FIG. 1B illustrates an embodiment of an inflatable retractor filled with a temperature reactive liquid or gas to inflate and deflate the retractor.

FIG. 2A-2C illustrates embodiments of a cylindrical shaped inflatable surgical retractor having a number of inflatable chambers.

FIG. 3A-3D illustrates embodiments of a conical shaped inflatable surgical retractor having a number of inflatable chambers.

FIG. 4A-4B illustrates embodiments of a helical shaped inflatable surgical retractor.

FIG. 5 illustrates embodiments of a finger shaped inflatable surgical retractor.

FIG. 6 illustrates a flow diagram for an exemplary embodiment of a method of using an inflatable retractor of the invention.

FIG. 7A illustrates an embodiment of a guide and drill used in installing an inflatable retractor of the invention.

FIG. 7B illustrates an embodiment of an inserter tool used in installing an inflatable retractor of the invention.

FIGS. 8A-8B illustrate another embodiment of a system for installing an inflatable retractor of the present invention.

FIGS. 9A-9D illustrate another embodiment of an inflatable retractor of the present invention using a hardening agent.

FIGS. 10A-10D illustrate another embodiment of a use of an inflatable retractor of the present invention.

### DETAILED DESCRIPTION OF THE INVENTION

The present invention provides an improved surgical device and method for retracting tissue from an incision and providing access to a surgical site in a patient. The inflatable body of the retractor allows the retractor to be inserted in a deflated or semi-inflated state and then inflated in-situ to effect retraction and provide access to the surgical site. Because the retractor is inserted in a non-inflated state, the incision used for insertion does not have to be as large as required to insert traditional retractors. The inflatable body of the retractor also allows the retractor to conform to the tissue and muscle as it retracts the tissue and muscle which causes less trauma to the patient. After the necessary surgical procedure is performed using the inflatable retractor, the retractor may be deflated and removed.

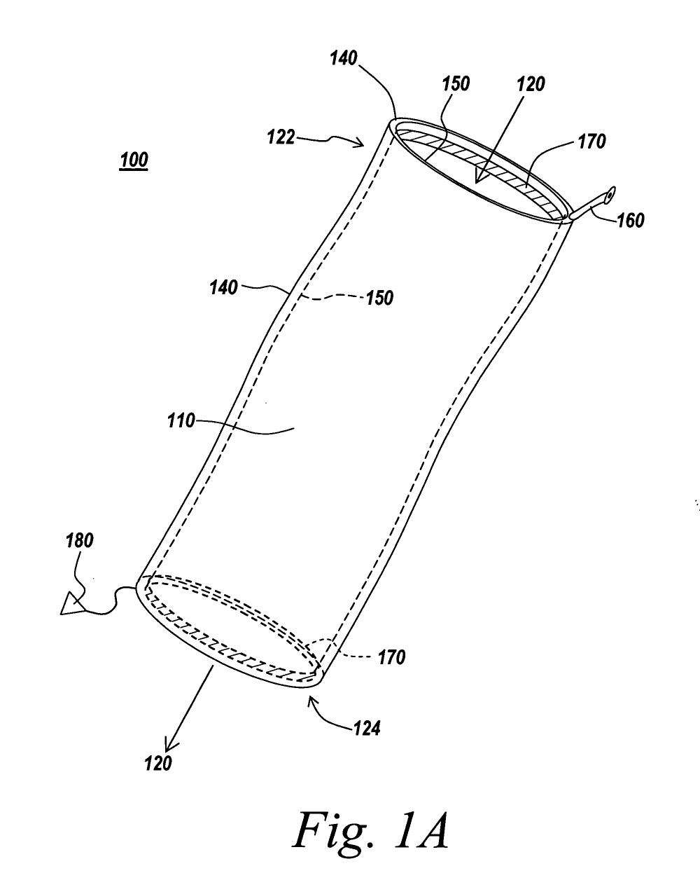

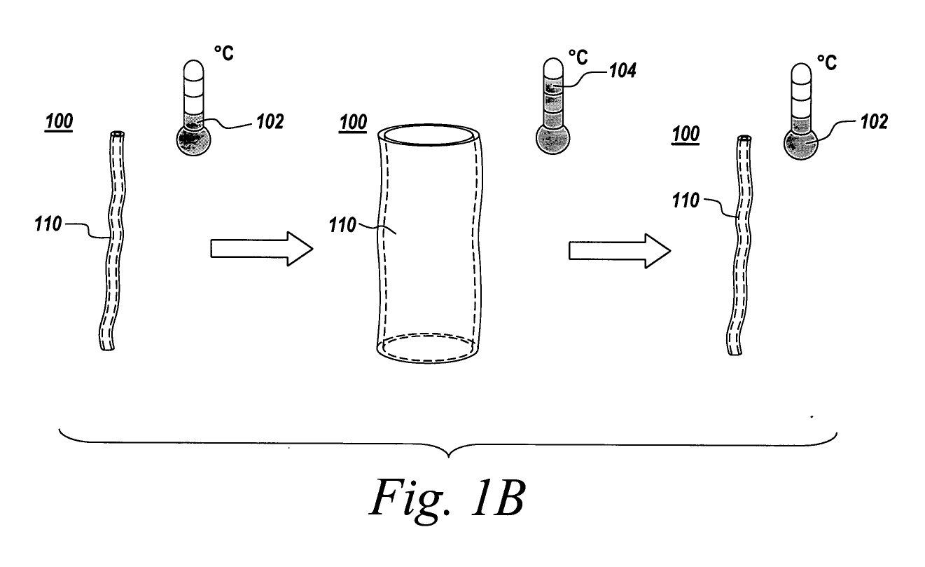

In one embodiment, shown in FIG. 1A, an inflatable retractor 100 comprises an inflatable body 110 defining a central cavity 120, wherein the body 110 is flaccid in a non-inflated state and increasingly rigid in an inflated state. In FIGS. 1A and 1B the inflatable retractor 100 is depicted in both an inflated state and a deflated state to illustrate the shape and configuration of the inflatable retractor 100.

In certain embodiments, the inflatable body 110 is dimensioned so that the defined cavity 120 provides a passage sized to access the spine of a patient, such as the vertebra. In some such embodiments the inflatable body 110 is dimensioned so the defined cavity 120 may provide a passage for posterior access to the spine of a patient. For example, the defined cavity 120 may have a diameter of 8-40 mm and a length of 30-200 mm. In other such embodiments, the defined cavity 120 may provide a passage for anterior access to the spine. Here, the defined passage may have a diameter of 150 mm and a length of 300 mm. The inflatable body 110 may be dimensioned to add 1-10 mm to the periphery of the defined passage. Other suitable configurations, dimensions, and applications will be apparent to one skilled in the art given the benefit of this disclosure.

To reduce adverse reactions with a patient when in use the inflatable body 110 may be formed of biocompatible material. Examples of suitable biocompatible materials include, but are not limited to, poly(ethylene terephthalate) and poly(etheretherketone). In some embodiments, a polymer or metal mesh may be embedded in the body 110 to improve puncture resistance. In certain embodiments, the outer surface 140 of the retractor 100 in contact with tissue may be formed of one material while inner surface 150 defining the cavity 120 is formed of another material. Other possible materials and combinations will be apparent to one skilled in the art given the benefit of this disclosure.

In some embodiments, the inflatable body 110 of the inflatable retractor 100 may be coated or impregnated with a therapeutic agent allowing the inflatable retractor 100 to be used for drug delivery at the surgical site. For example, pain medication, antibiotics, anti-inflammatories, and other medications may be applied to or impregnated in the inflatable body 110, or applied to the outside of the body 140 and delivered at the incision site by the inflatable body 110.

In some embodiments an illuminant can be coupled to the inflatable body 110 to provide illumination at the incision site. In one example, the illuminant may be a luminescent chemical coupled to the interior surface 150 defining the cavity. In one such embodiment, the interior surface 150 is treated with a luminescent chemical. In another such embodiment, inflatable body 110 may be formed of a luminescent material. In another example, the inflatable body 110 may be formed of a translucent material and filled with a luminescent chemical, or the body may be formed of a translucent material and have a provision to attach to a light generating source separate from the retractor.

In certain other embodiments, the inflatable body 110 of the retractor 100 may include radiopaque markers. These markers aid in the percutaneous insertion of the retractor 100 under fluoroscopy.

In some embodiments, the inflatable retractor 100 further includes a valve 160 functional to maintain an inflating pressure in the inflatable body 110. A gas, liquid, or combination of both may be introduced through valve 160 to inflate the inflatable retractor 100. Suitable inflating mediums include, but are not limited to water, saline solution, air, and the like. In certain embodiments the valve 160 may also be used to release the inflating pressure thereby deflating the inflatable body 110. In some such embodiments, the retractor 100 may be inflated and deflated multiple times allowing the inflatable retractor 100 to be re-used, or the pressure adjusted interoperatively to relieve pressure on the skin, tissue, and nerves.

The required inflation pressure depends on the use of the inflatable retractor 100 as well as the type and amount of tissue being retracted. For most applications, a pressure of 80 psi would be suitable to inflate the body 110 and provide retraction. It should be understood that the type of material the inflatable body is formed of as well as the inflation medium may also affect the inflation pressure.

In some embodiments, the inflatable body 110 may be filled with a temperature sensitive liquid or gas. In one such example, the inflatable body 110 could be filled with a hardening agent, like an epoxy, that sets-up or solidifies and makes the inflated retractor rigid. In one such example, the liquid may be a wax-like material that can be made to harden or expand at a certain temperature to rigidify or inflate the retractor 100 and then re-liquefy by changing the temperature to collapse or deflate the retractor 100. In the example of a temperature sensitive gas, the gas could expand at certain temperature, such as internal body temperature, to inflate the retractor and contract at another temperature, such as room temperature, to deflate the retractor 100. An example of this can be seen in FIG. 1B. Here the retractor 100 is deflated at a first temperature 102, such as room temperature or a cooled state. As the temperature is increased to a second temperature 104, the liquid or gas in the body 110 expands to inflate the retractor 100. Returning the temperature to the first temperature 102 deflates the body. The temperatures at which the retractor 100 may expand or contract may be set as needed by selecting the appropriate gas or liquid with the desired temperature characteristics. The temperature, and thus the inflation, may be controlled by externally applying fluids or gases or other heating or cooling means as needed. Other possible implementations will be apparent to one skilled in the art given the benefit of this disclosure.

Returning again to FIG. 1A, in some embodiments, the inflatable retractor 100 may further include a support structure 170 for maintaining the shape of the inflatable body. For example, inflatable body may be a spline or ring 170 of shape memory material, such as Nitinol, that can be collapsed for insertion and then be expanded once the retractor is deployed to help maintain the shape of the inflatable body 110 under load. Other configurations and implementations will be apparent to one skilled in the art given the benefit of this disclosure.

In some embodiments, the inflatable retractor 100 may further include an anchor 180 attached to the inflatable body 110 for anchoring the inflatable body to a body part within the incision. The anchor 180 may comprise any suitable device such as a screw, pin, wire, or the like for anchoring the inflatable body 110 to a body part. For example, in one embodiment, the anchor 180 comprises a resorbable anchor. Alternatively the anchor 180 may comprise an anchor formed of a bio-compatible non-resorbable material. The anchor 180 may attach directly to the bone or body part. Alternatively, an anchor that is left behind after the retractor is removed and serves a second purpose such as pedicle screws fixed to the pedicle bone and used for supporting instrumented spinal stabilization, may be used to connect an inflatable body 110 to the body part.

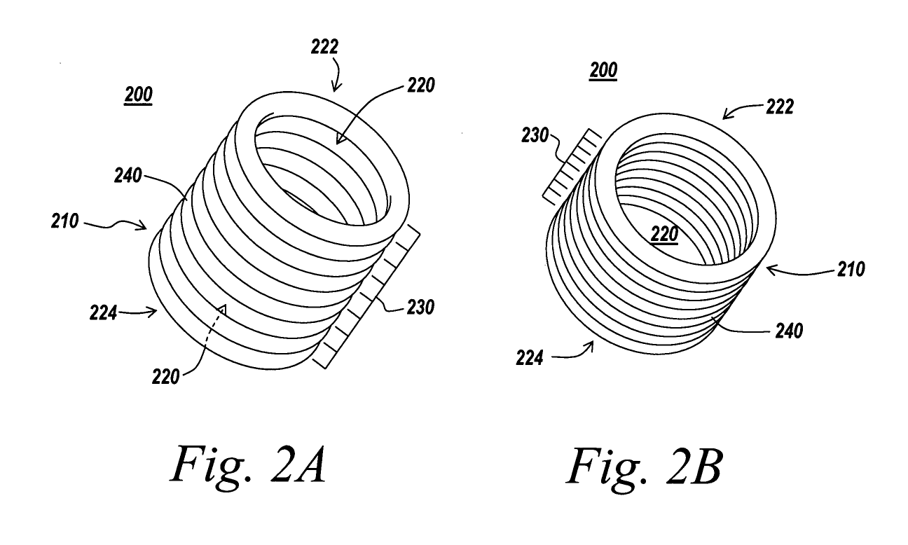

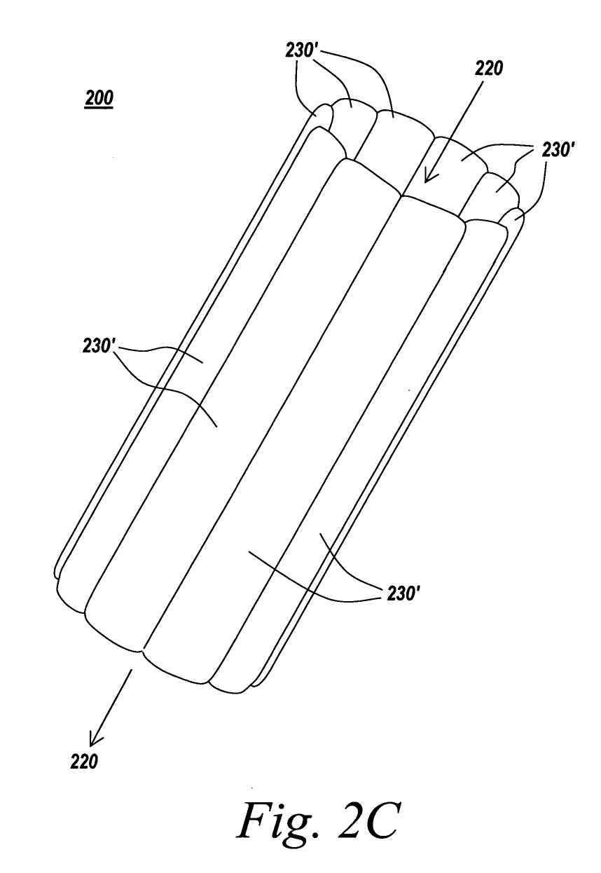

In some embodiments as seen in FIGS. 2A-C, the inflatable body 210 may be comprised of one or more inflatable chambers 230. The use of multiple chambers 230 can reduce the likelihood of a puncture causing total deflation of the retractor 200. For example the multiple chambers 230 can be connected by one-way valves allowing for inflation but keeping the retractor from completely deflating if one chamber 230 is punctured. In FIGS. 2A-C the inflatable retractor is depicted in an inflated state to better illustrate the shape and configuration of the inflatable retractor. In the example of FIGS. 2A and 2B, the multiple chambers 230 form a series of rings around the circumference of the defined cavity 220. Alternatively, the multiple chambers 230′ can be formed along the length of the defined cavity 220 as shown in FIG. 2C.

In addition to increased puncture resistance, the curvature of the outer surface 240 of the retractor 200 formed by the multiple chambers 230, 230′ provides increased surface area traction with the tissue and muscle at the surgical site. This assists in both the retraction of the tissue and muscle by the retractor 200 as well the anchoring or fixation of the retractor 200 at the surgical site. Additionally, this increased surface area spreads the load more evenly on the tissue, reducing areas of high pressures. In some embodiments, the outer surface 240 of the retractor 200 may be textured to further increase traction. In some embodiments, the inflatable retractor 200 may also have distal protrusions for assisting in the retraction of tissue and minimizing tissue creep.

In the embodiment of the retractor depicted in FIGS. 1-2C, the inflatable body is cylindrical in shape and defines a cavity through the body which may provide a passage for accessing the patient. Thus, when deployed in a patient, the retractor 100, 200 provides an access port from outside of an incision at a proximal end 122, 222 of the defined cavity 120, 220 to a surgical site at the distal end 124, 224 of the defined cavity 120, 220. It should be understood however, that cylindrical is but one possible shape and there are any number of other possible shapes and configurations.

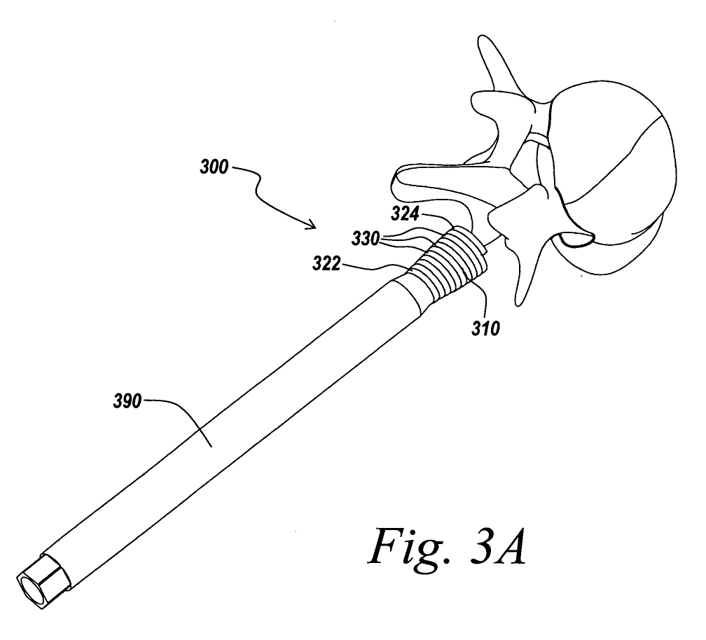

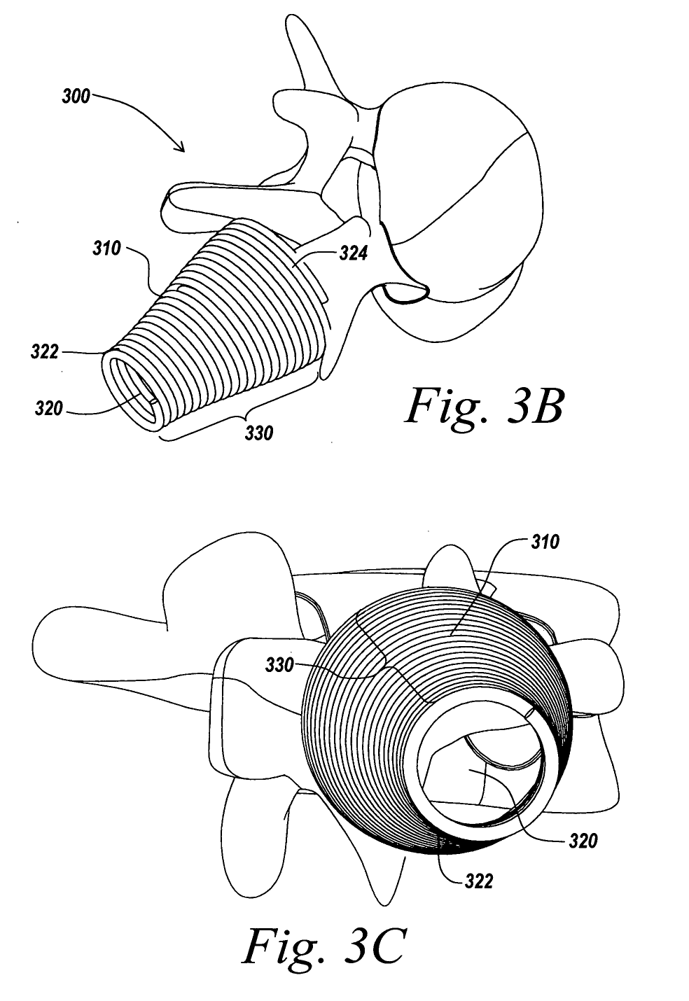

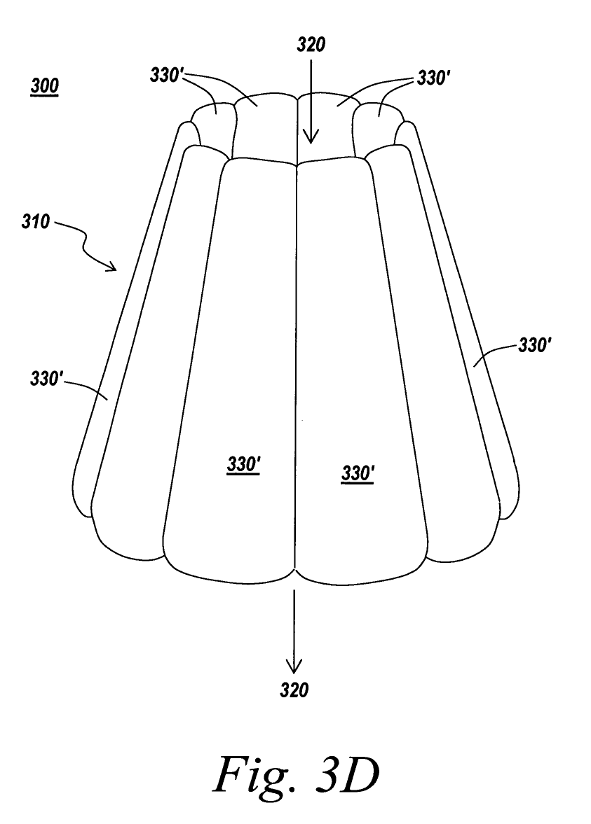

In another embodiment of an inflatable retractor 300 as shown in FIGS. 3A-D, the inflatable body 310 is conical in shape and defines a cavity 320 though the body 310 that provided a passage for accessing a patient. Thus, when deployed in a patient, the inflatable retractor 300 provides a body that is larger at the distal end 324 of the defined passage 320 then at the proximal end 322 of the defined cavity 320. FIG. 3A illustrates the inflatable retractor 300 being delivered to a surgical site, i.e. a vertebral body, on the end of an insertion instrument 390. As with the example in FIGS. 2A and 2B, the inflatable body 310 in FIGS. 3A-C comprises multiple chambers 330 that form a series of rings around the circumference of the defined cavity 320. As with FIG. 2C, the multiple chambers 330′ of FIG. 3D are formed along the length of the defined cavity 320.

The conical shape provides some additional benefits when deployed in a patient. One benefit is that the larger opening at the distal end 324 of the cavity 320 provides a larger access area at the surgical site than provided by cylindrical shaped retractor without requiring a larger incision. The flared nature of the conical shaped retractor 300 also assists in the anchoring or fixation of the retractor 300 at a surgical site because the retracted tissue and muscle is displaced in such a way that it creates a partial downward pressure on the retractor walls, rather than only a normal pressure on the walls as would be the case in a cylindrical retractor.

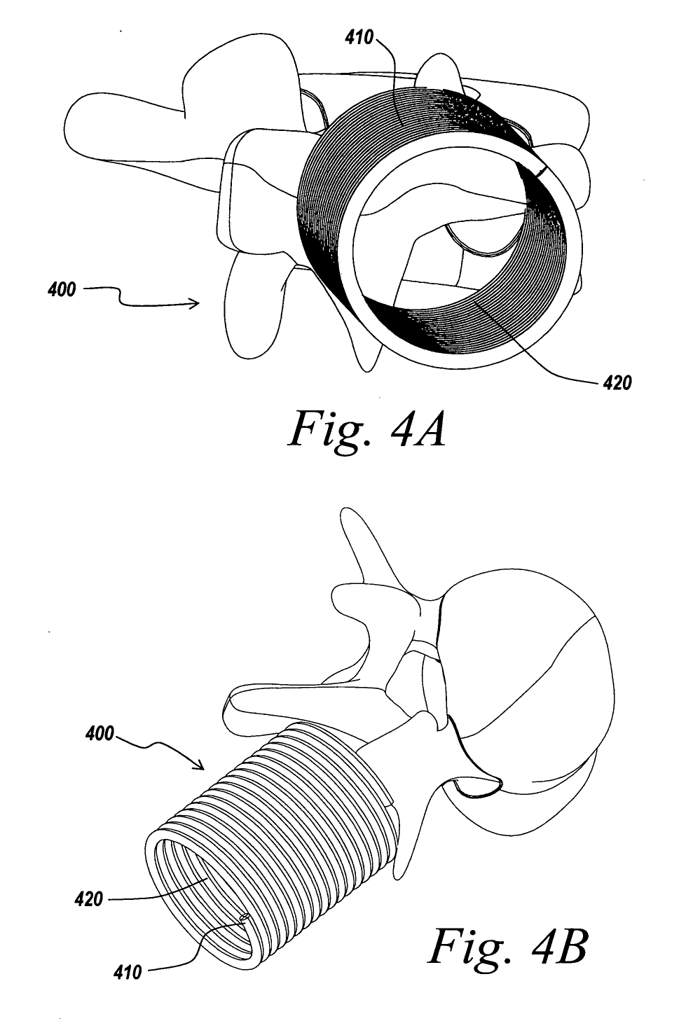

In another embodiment of the inflatable retractor 400 of the present invention, as shown in FIGS. 4A and 4B, the inflatable body 410 is helical in shape and configured to be coiled upon itself to define a cavity 420 for accessing a patient. In use, the inflatable body 410 can be inserted in a patient in a deflated or semi-inflated state. One advantage of a helical shape is that the inflatable body 410 can be configured as needed to define a cavity 420 to provide access to a surgical site. Thus, while the helical shape can be configured in a cylindrical shape or conical shape as show in FIGS. 2A-C and 3A-D, it is not limited to a fixed depth or cavity width of the fixed-shaped cylindrical or conical shaped retractors 200, 300 of FIGS. 2A-C and 3A-D.

While the above examples of inflatable retractors depict specific configurations (cylindrical, conical, and helical) it should be understood that the inflatable body of the inflatable retractor can assume any number of possible shapes and configurations. Indeed, the conformable nature of the inflatable body allows it to be configured and used in other applications. An example of this can be seen in FIG. 5.

In FIG. 5, the inflatable retractor 500 is configured to mimic a finger. As with the previously described inflatable retractors, the inflatable retractor of FIG. 5 has an inflatable body 510 defining a cavity 520 for providing access to a patient. In this example, the inflatable body 510 is configured to be generally finger-like in shape. The inflatable retractor may further include a rigid shaft 590 for manually manipulating the inflatable body 510 as it is inserted into the incision.

In many instances, a surgeon will use a finger inserted into the incision to probe and perform blunt dissection between the tissue and muscles planes of a patient. The soft conforming nature of the inflatable retractor 500 allows the retractor to be used to probe and dissect the tissue and muscle planes in place of a finger. Traditionally, once the probing and separation are performed, the surgeon must insert a dilator, retractor or similar tool into the incision in place of the finger to affect the actual retraction. The advantage of using an inflatable retractor for the probing and separation, is that once the probing and separation are completed the inflatable retractor 500 can then be used to dilate and/or retract the tissue without requiring it to be replaced with another instrument.

In another embodiment, the surgeon may place their finger inside the retractor 500 in a non-inflated state, and use his or her finger, now encased in the retractor, to probe and dissect the tissue. When the surgeon removes their finger the retractor will remain in place and can be inflated to retract the tissue without being replaced by an additional instrument.

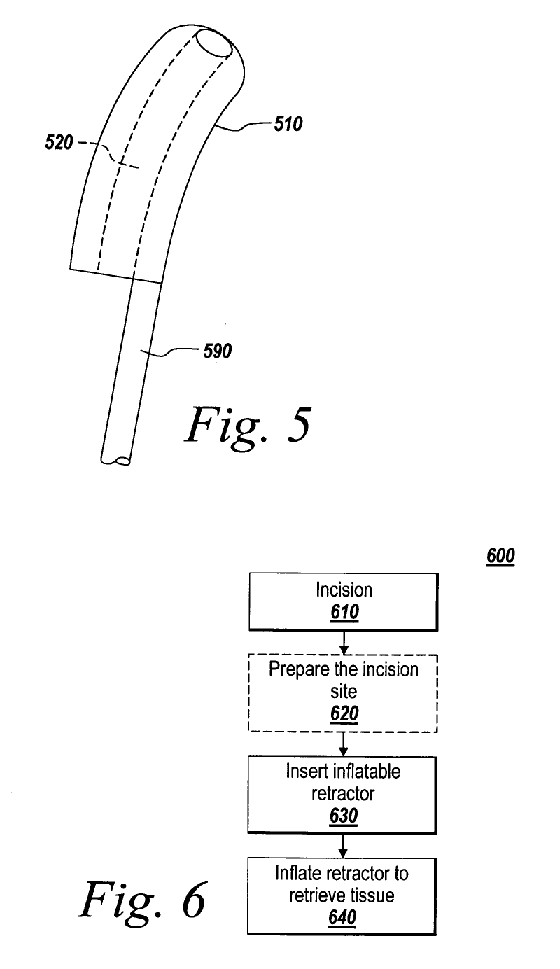

FIG. 6 is a flow diagram 600 for one exemplary method of providing access to a surgical site in a patient using an inflatable retractor. First, an incision is made at the surgical site 610. The incision site may then be prepared for the insertion of the inflatable retractor 620. The inflatable retractor can then be inserted into the incision site 630. Once inserted, the inflatable retractor can then be inflated to retract tissue from the surgical site 640.

In one embodiment, the step of preparing the incision (step 620) involves additional steps. For example, blunt finger dissection between muscle planes may be performed using the actual finger of the surgeon or the inflatable retractor 500 of FIG. 5. Continuous or serial dilation with or without a guide-wire may be performed to expand the incision opening for insertion of a guide or retractor. Alternately, the inflatable retractor itself may be used for dilation.

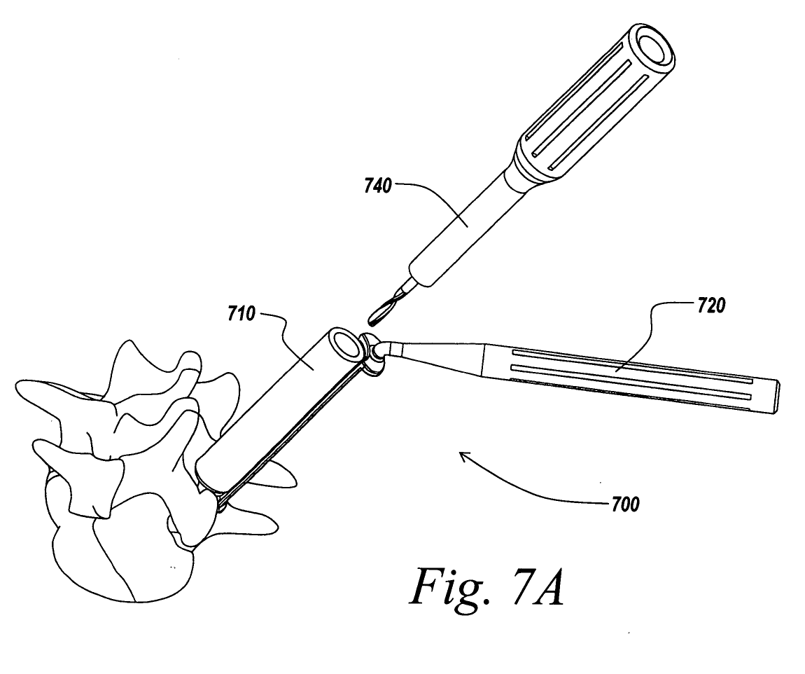

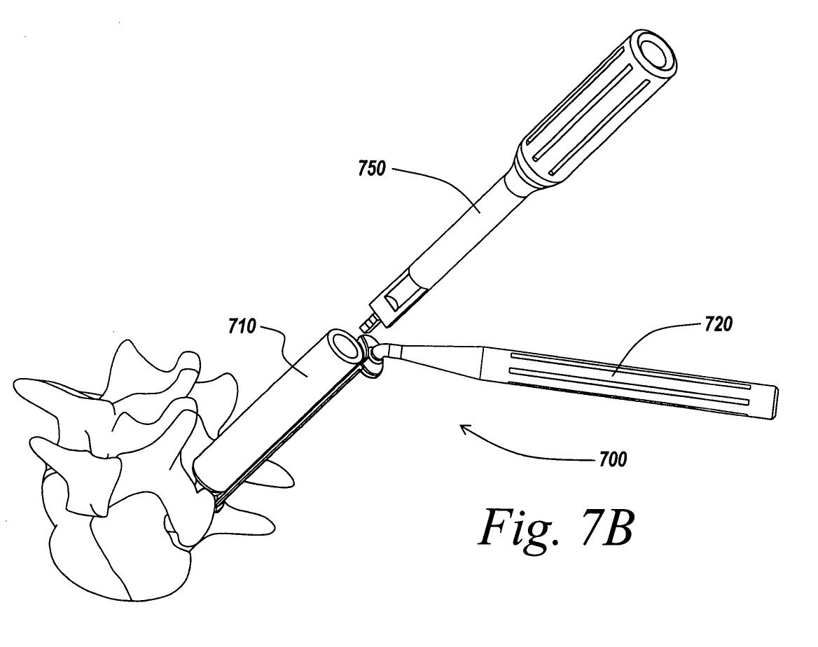

Once dilation is completed, then a guide can be inserted into the incision. The guide can be used to sweep back muscle and tissue from the surgical site to find a target for inserting the inflatable retractor. An example of suitable guide can be seen in FIG. 7A. Here the guide 700 features a hollow insertion cannula 710 for guiding the insertion and removal of surgical instruments, including the inflatable retractor, and a handle 720 attached to the proximal end of the cannula 710 for inserting and positioning the insertion cannula 710 in the incision site. Once a target for placing the inflatable retractor is located a drill 740 or awl may be used to create an anchor hole for attaching the anchor attached to the inflatable body. Examples of suitable targets include portions of a vertebra, such as a pedicle, lamina, vertebral body, spinous process, transverse process, or the like. In certain embodiments intermediate anchors such as pedicle screws can be used for attaching the inflatable body.

Referring again to FIG. 6, the step of inserting the inflatable retractor (step 630) may also involve multiple steps. In one example, these steps include attaching an anchor on a distal end of the inflatable retractor to a bone in the surgical site through the incision. In some embodiments this involves using the guide shown in FIG. 7. In such an example, the guide, such as a hollow insertion cannula, is inserted in the incision. An anchor of the retractor is passed through the hollow insertion cannula such that the body of the retractor in a deflated state extends through an opening in the hollow insertion cannula. The anchor of the retractor is then attached to the target. The hollow insertion cannula may then be withdrawn so that the deflated or semi-inflated retractor extends through the incision. In some embodiments an insertion tool 750 as shown in FIG. 7B is used. The inserter tool 750 is used to pass the anchor of the inflatable retractor though the insertion cannula and attach the anchor to the target.

The inflatable retractor is preferably inserted into the incision in a deflated state. In some embodiments, the deflated retractor is inserted using an instrument such as a dilator, catheter, trochar, or sleeve. In the case of a dilator, the deflated retractor may be wrapped around the dilator which is inserted into the incision. When the dilator is withdrawn from the incision the retractor unwraps from the dilator and is left behind in the incision. In another embodiment, the deflated or semi-inflated retractor may be inserted along a guide-wire. In still other embodiments, the deflated or semi-inflated retractor may be inserted and positioned by using a finger of the surgeon as discussed above. Alternately, a catheter, trochar, or sleeve can be used to contain, insert, and deploy the retractor in a deflated state. In some embodiments, additional instruments, such as forceps, may be used to position and deploy the inflatable retractor in situ. In some such embodiments, the positioning of the retractor in situ is performed with the aid of radiopaque markers that are discernable when viewed on a fluoroscope.

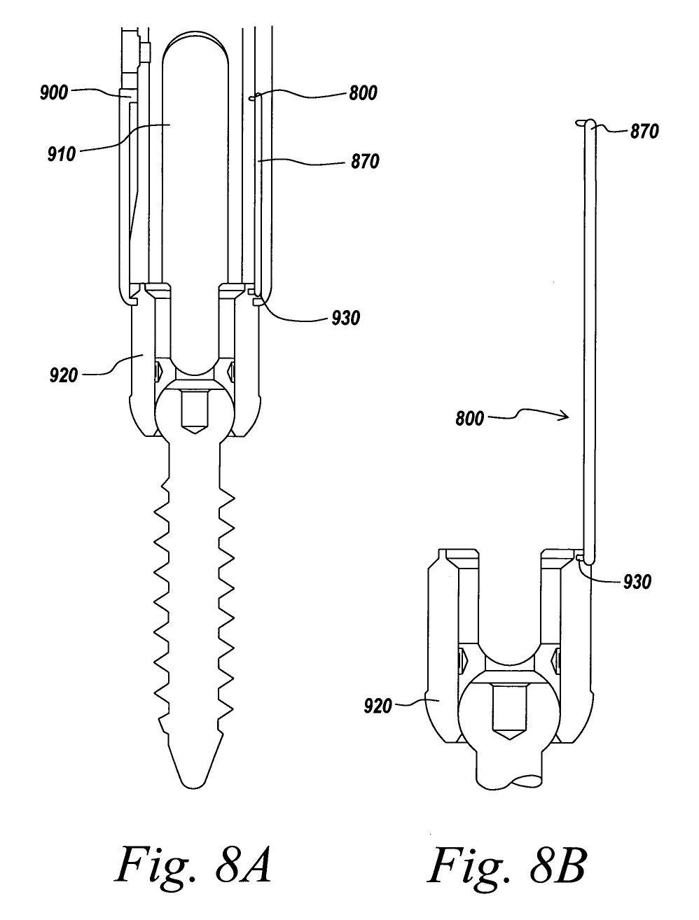

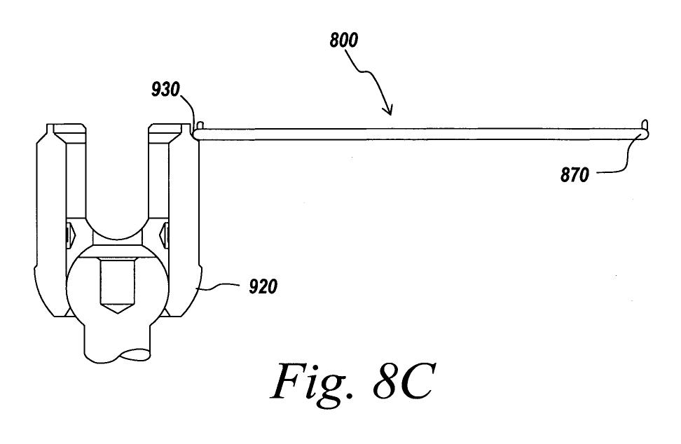

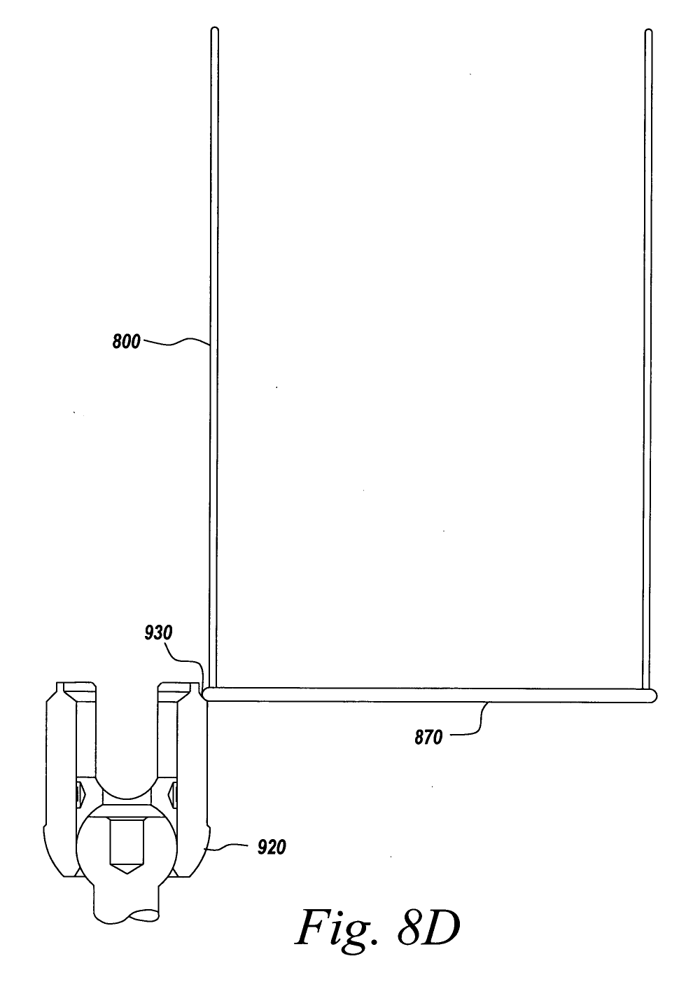

In another embodiment, as depicted in FIGS. 8A and 8B, the retractor 800 could be inserted by being contained in the sheath 900 of a cannulated screwdriver 910 while attached to a bone screw 920 as seen in FIG. 8A. Once the bone screw 920 is in place, the sheath 900 can be removed leaving the deflated retractor 800 attached to the screw 920 as seen in FIG. 8B. The retractor 800 may then be deployed by rotating it into position, as seen in FIG. 8C, and inflating the retractor 800 as seen in FIG. 8D. In this particular embodiment, the retractor 800 further includes a support ring 870 to maintain the shape of the inflatable body 810. The retractor 800 may be attached to the screw by a pin, c-clip, retaining ring, polymer tether, fracture tab or other suitable removable attachment means 930.

Once the inflatable retractor has been inserted, it can then be inflated to retract tissue at the surgical site (step 640). As discussed above, the inflatable retractor may be inflated with a liquid, gas, or combination of both.

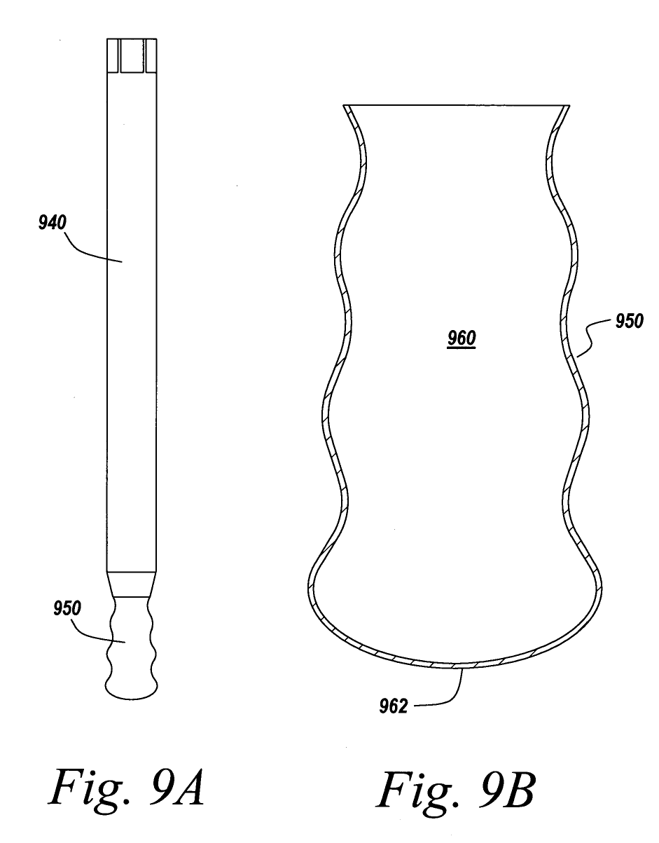

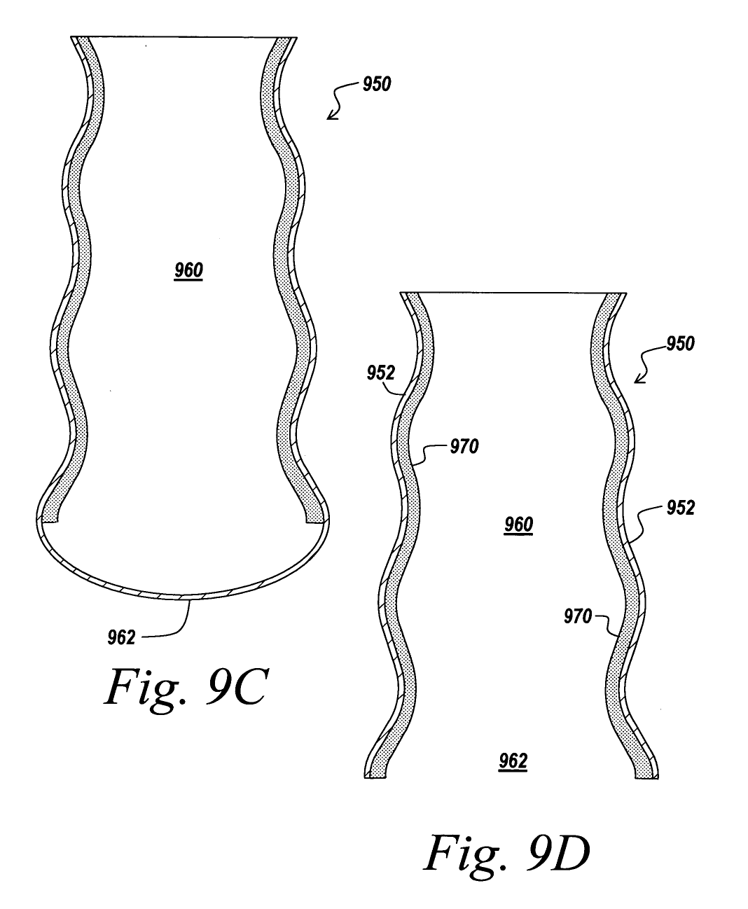

In some embodiments a hardening agent, such as an epoxy, may be used inflate the retractor. Once the retractor is inflated the epoxy sets-up or hardens to make the retractor rigid. Alternately, a hardening agent, such as a spray epoxy, may be applied to the inner surface of the retractor inflated with gas or liquid to rigidify the retractor. An example of this can be seen in FIGS. 9A-9D.

In FIG. 9A an insertion device 940 is used to deploy an inflatable body 950 at a surgical site. Once deployed at the surgical site, the inflatable body 950 may be inflated to retract tissue and create a cavity 960 for accessing the surgical site as seen in FIG. 9B. In this embodiment, the inflatable body 950 is sealed at the distal end 962 of the cavity 960 for deployment and inflation. Once the inflatable body is inflated a hardening agent 970 may be applied to the interior of the cavity 960 such that the sidewalls 952 are coated with the hardening agent 970 as seen in FIG. 9C. Once the hardening agent 970 has set up such that the sidewalls 952 of the inflatable body 950 defining the cavity 960 are rigid, the distal end 962 of the cavity 950 may be opened to provide access to the surgical site as shown in FIG. 9D.

Once sufficient retraction of issue is obtained, necessary surgical procedures may then be performed at the surgical site. After the procedure has been performed at the surgical site, the inflated retractor may be deflated by puncturing the inflated body with an instrument such as a scalpel or releasing the pressure valve, or changing the temperature of the internal media, or by adding a softening agent. If the retractor includes an anchor, the anchor may be disconnected. The deflated retractor may then be removed and the incision closed. As discussed above, the anchor may be made of a reabsorbable or biocompatible material such that the anchors may be left in the body of the patient.

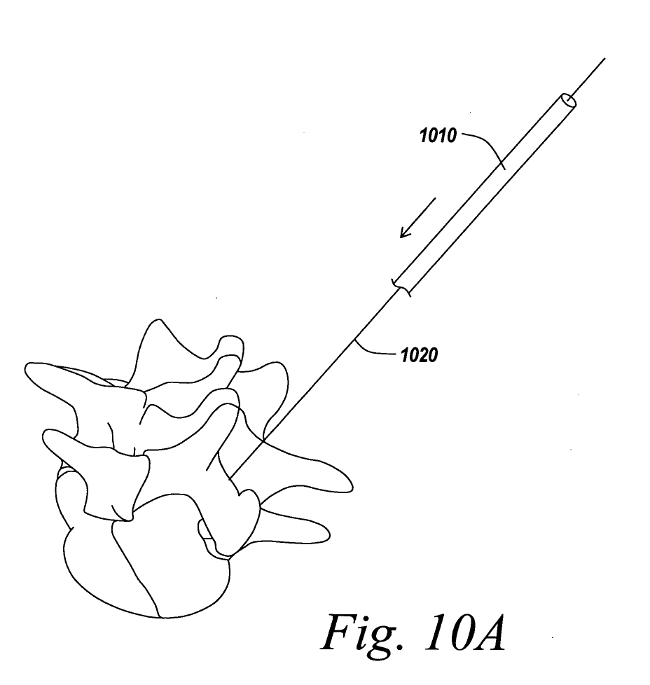

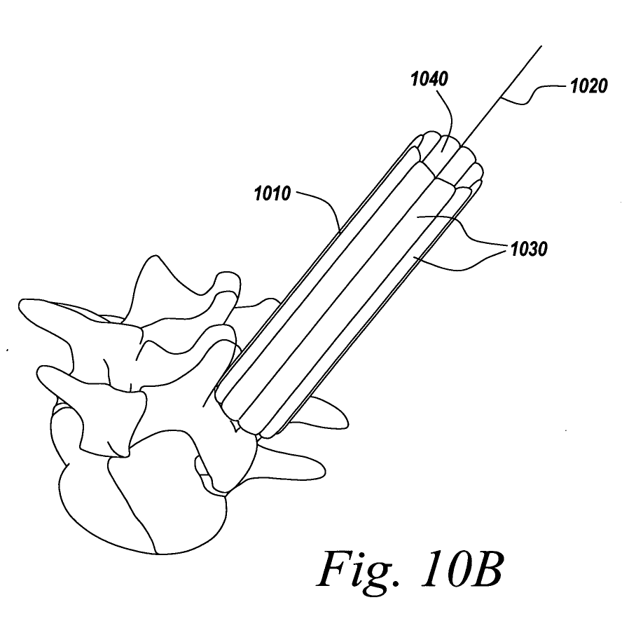

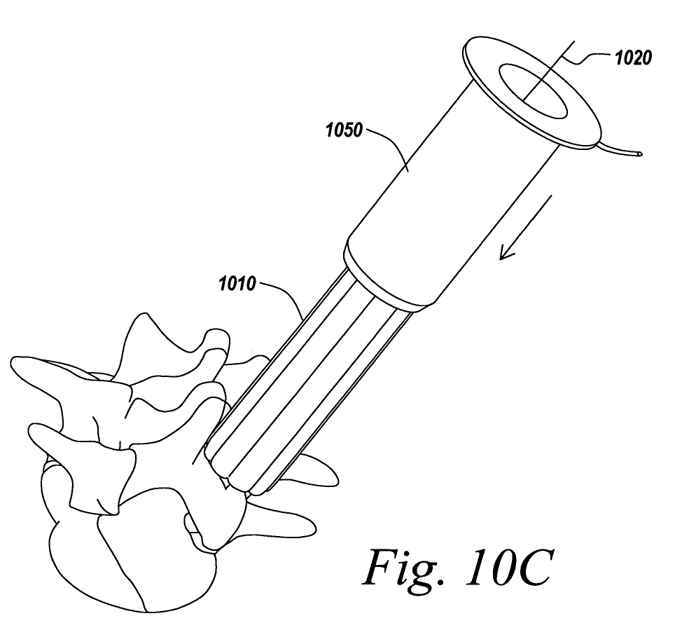

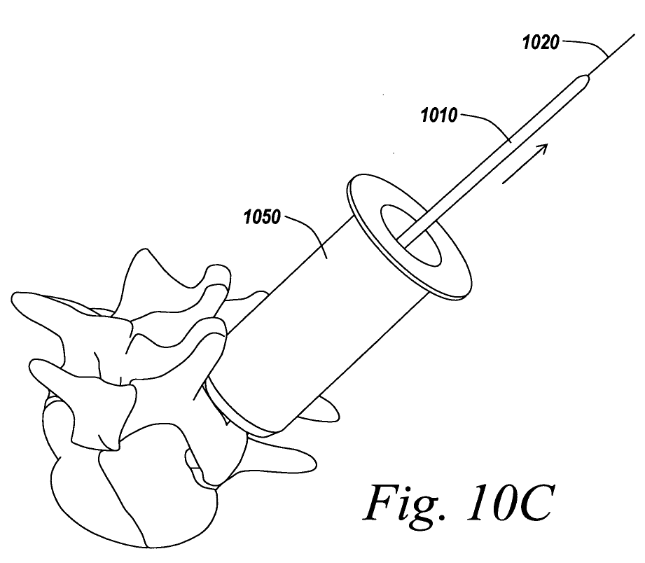

In some embodiments, such as when the inflatable retractor is used for dilation, an access device such as a rigid port, expandable retractor, or traditional retractor may be inserted over the inflatable retractor to maintain access to a surgical site. An example of this can be seen in FIGS. 10A-D. Here, as shown in FIG. 10A, an inflatable body 1010, in a deflated or semi-inflated state, is inserted over a guide-wire 1020 to a surgical site. Alternately, the inflatable body 1010 may be inserted using any of the techniques discussed herein. Other possible insertion techniques will be apparent to on skilled in the art given the benefit of this disclosure.

Once the inflatable body 1010 is in position, the inflatable body may be inflated to perform dilation and/or retraction of tissue at the surgical site. An example of this can be seen in FIG. 10B. In this example the inflatable body 1010 is cylindrical in shape with multiple chambers 1030 formed along the length of the defined cavity 1040. Other possible configurations will be apparent given the benefit of this disclosure. The inflatable body may be inflated using any of the techniques that have been described above. The guide-wire 1020 may also be removed after inflation.

Once the inflatable body 1010 is inflated and the incision and tissue dilated and retracted an access device, such as a rigid port 1050 may be inserted over the inflatable body 1010. An example of this can be seen in FIG. 10C. In this example, the access device is a port such as ports available in the Pipeline Access System distributed by DePuy Spine (Raynham, Mass.). In other embodiments, the access device may be a retractor such as the Pipeline Expandable Retractor distributed by DePuy Spine (Raynham, Mass.). Other suitable rigid access devices will be apparent to one skilled in the art given the benefit of this disclosure.

After the access device 1050 is in position, the inflatable body 1010 may then be deflated and removed as discussed above. An example of this can be seen in FIG. 10D. With the rigid access device 1050 now providing access to the surgical site, any necessary procedures may then be performed through the cavity of the access device 1050.

The apparatus and techniques of the present invention provide numerous advantages. The inflatable retractor of the present invention can be used in any approach, including lateral, posterior, and anterior. The inflatable retractor is compact in a deflated state allowing for insertion through a smaller incision and requiring little or no dilation of the incision before insertion. The flexible nature of the inflatable body prevents damage to tissue, vessels, and muscle. The ability to anchor the retractor internally alleviates the need for a bed-mounted arm to secure the retractor.

The present invention has been described relative to an illustrative embodiment and application in spinal correction surgery. It should be apparent that the present invention may be used in any number of surgical procedures. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

1. An inflatable retractor for retracting tissues in an incision for a surgical site, comprising:
an inflatable body defining a cavity, wherein the body is flaccid in a non-inflated state and increasingly rigid in an inflated state.

2. The inflatable retractor of claim 1, wherein the cavity provides a passage for accessing a patient.

3. The inflatable retractor of claim 1, wherein the inflatable body is formed of a biocompatible material.

4. The inflatable retractor of claim 1, wherein the inflatable body comprises one or more inflatable chambers.

5. The inflatable retractor of claim 1, further comprising a valve functional to maintain an inflating pressure in the inflatable body.

6. The inflatable retractor of claim 1, further comprising an illuminant coupled to the inflatable body for illuminating the surgical site.

7. The inflatable retractor of claims 1, further comprising a therapeutic agent coating on the inflatable body.

8. The inflatable retractor of claim 1, wherein the inflatable body is cylindrical in shape.

9. The inflatable retractor of claim 1, wherein the inflatable body is conical in shape.

10. The inflatable retractor of claim 1, wherein the inflatable body is helical in shape and configured to be coiled upon itself to define a passage for accessing a patient.

11. The inflatable retractor of claim 1, wherein the inflatable body is shaped like a finger.

12. The inflatable retractor of claim 1 further comprising a support structure for maintaining the shape of the inflatable body.

13. The inflatable retractor of claim 12, wherein the support structure is formed of a shape memory material.

14. The inflatable retractor of claim 1 further comprising an anchor attached to the inflatable body for anchoring the inflatable body to a body part within the incision.

15. The inflatable retractor of claim 1 wherein the surgical site comprises a patient's spine.

16. The inflatable retractor of claim 1 wherein the inflatable body comprises a puncture resistant material.

17. A method of accessing a surgical site in a patient comprising the steps of:
creating an incision in the patient,
inserting an inflatable retractor into the incision in the patient, the inflatable retractor comprising an inflatable body defining a central cavity, wherein the body is flaccid in a non-inflated state and increasingly rigid in an inflated state; and
inflating the inflatable retractor to retract tissue surrounding the incision.

18. The method of claim 17, further comprising the step of preparing the incision prior to inserting the inflatable retractor.

19. The method of claim 18, wherein the step of preparing the incision comprises the steps of:
inserting a guide into the incision; and
sweeping back muscle and tissue from the surgical site; and
finding a target for attaching an inflatable retractor.

20. The method of claim 17, wherein the step of inserting the inflatable retractor comprises using an instrument to insert the inflatable retractor into the incision.

21. The method of claim 17, wherein the step of inserting the inflatable retractor comprises using a finger to insert the inflatable retractor into the incision.

22. The method of claim 17, wherein the step of inserting the inflatable retractor is assisted by the use of radiopaque markers in the retractor.

23. The method claim 17, further comprising attaching the inflatable retractor to a target.

24. The method of claim 17, wherein the step of inserting the inflatable retractor comprises the steps of:
inserting a hollow insertion cannula in the incision;
passing an inflatable retractor through the hollow insertion cannula;
attaching the inflatable retractor to a target; and
removing the hollow insertion cannula.

25. The method of claim 17, wherein the step of inserting the inflatable retractor comprises the steps of:
inserting a guide-wire in the incision;
inserting the inflatable retractor over the guide-wire; and
removing the guide-wire

26. The method of claim 17 further comprising the steps of:
deflating the inflatable retractor; and
removing the inflatable retractor from the incision.

27. The method of claim 17, further comprising the steps of inserting a access device over the inflated retractor.

28. The method of claim 17 wherein the retractor is inflated with a gas.

29. The method of claim 17 wherein the retractor is inflated with a liquid.

30. The method of claim 17 wherein the retractor is inflated with a hardening agent.

31. The method of claim 17 further comprising applying a hardening agent to the inflated retractor.

32. A system for retracting tissues in an incision for a surgical site, the system comprising:
an insertion tool for insertion into the incision at a surgical site; and
inflatable retractor configured to be guided to a surgical site by the insertion tool, the inflatable retractor comprising an inflatable body defining a central cavity, wherein the body is flaccid in a non-inflated state and increasingly rigid in an inflated state

33. The system of claim 32, wherein the insertion tool comprises a guide-wire and the inflatable retractor is guided to the surgical site over the guide-wire.

34. The system of claim 32, wherein the insertion tool comprises a cannula and the inflatable retractor is guided to the surgical site through the cannula.

35. The system of claim 32, further comprising an access device configured for insertion over the inflatable retractor.

36. The system of claim 35, wherein the access device is a rigid port.

37. The system of claim 35, wherein the access device is a retractor.
